(19) United States
(12) Patent Application Publication
(10) Pub. No.: US 2020/0372179 A1
(43) Pub. Date: Nov. 26, 2020

(54) A METHOD AND APPARATUS FOR SECURING HEALTH DATA

(71) Applicant: SAFE2HEALTH PTY LIMITED, Sydney, New South Wales (AU)

(72) Inventors: Admir Nasic, Mascot, New South Wales (AU); Schaan O'Hara, Willoughby, New South Wales (AU)

(21) Appl. No.: 16/316,081

(22) PCT Filed: Jul. 10, 2017
(86) PCT No.: PCT/AU2017/050709

§ 371 Date: Jan. 8, 2019

(30) Foreign Application Priority Data
Jul. 8, 2016 (AU) 2016902694

(51) Int. Cl. G06F 21/62; H04L 9/32; G16H 10/60

(52) U.S. Cl. CPC G06F 21/6254; H04L 9/3213; H04L 2209/42; G06F 2221/0711; G16H 10/60

(57) ABSTRACT

The present invention relates to a method, apparatus and system for securing health data for transportation over communications network. It is important that heath data of a patient be kept secure. The apparatus and system of this invention secure health data for transport by preparing the health data in an anonymous form and associating a token with the health data. The token is used to secure the health data and also to enable access to the health data.

### FIELD OF THE INVENTION

The present invention relates to a method, apparatus and system for securing health data, and, particularly, but not exclusively, to a method, apparatus and system for securing health data for transportation over a communications network.

### BACKGROUND OF THE INVENTION

It is important that information relating to the health of a patient be generally secure. The patient usually desires that their health information be kept private. Doctors are usually under legal or regulatory obligations not to share patient data without a patient's permission.

With more and more health information being kept as data on computing systems, it is necessary to ensure that the computing systems are secure. Unfortunately, computing systems are open to clever hackers “cracking” the system and obtaining data. Health data is also vulnerable when it is being transmitted across computer networks and is subject to potential hacking.

### SUMMARY OF THE INVENTION

In accordance with a first aspect, the present invention provides a method of securing health data for transport from an originating system to a destination system over a communication network, comprising the steps of preparing the health data in an anonymous form, associating a token with the health data for transmission with the health data, associating a token with a patient identity at the destination system, receiving the health data and its associated token at the destination system, comparing the token associated with the health data with the token associated with the patient identity and, if they match, associating the patient identity with the health data.

In an embodiment by “anonymous form”, is meant that the health data does not include and is not associated with any patient identifier or anything that could be used to identify the patient. The “patient identifier” may be anything that could possibly be used to identify the health data. In an embodiment it may be the name of the patient, the identity of the patient device which is being used to generate health data, the address of the patient, or any other identification data.

A token may be any token. It could, for example, be a number, a picture, letters, a combination of numbers, letters, pictures, any code, or any other form of token.

In an embodiment, the health data is only associated with a patient identity when it reaches the destination system. During transport over the communications network (“in flight”) the health data is unidentified (anonymous). It is therefore mainly useless to anyone who should obtain the information (e.g. a hacker). Health data without an associated patient identification is of no real use.

In an embodiment, when health data is to be transported, the destination system generates a token to associate with the patient identity which the health data relates to, and sends a copy of the token to the originating system. At the originating system, the copy of the token can be associated with the anonymized health data for transmission to the destination system. On receipt of the data the destination system matches the copy token with the token associated with the patient identity to confirm the identity for the health data.

In an alternative embodiment, a token is generated at the originating system and a copy is transmitted to the destination system. A generated token is associated with the health data. When the destination receives the health data and token, it matches the associated token with the copy (which is associated with the patient identity) to confirm patient identity.

In an embodiment, the step of preparing the health data comprises the step of preparing the health data as a plurality of data packets for separate transmission. In an embodiment, a different token is associated with each data packet. In an embodiment, a corresponding plurality of tokens are associated with the patient identifier at the destination system. This arrangement advantageously increases security of the health data. Because the data is divided into separate packets, which are transmitted separately, it would be almost impossible for a hacker to obtain all the packets and hack them. A different token being provided for each packet makes it even more difficult for a hacker to be able to obtain any relevant or useful information at all.

In an embodiment, the method comprises the further step of storing the health data. The step of storing comprises associating a token with the health data, after anonymizing the health data, so it is stored in a tokenized and anonymous form. A hacker breaking into the storage system, would find it impossible or at least very difficult to obtain any information on the identity of the patient associated with the health data. In an embodiment, matching tokens are stored elsewhere and associated with patient identities. The health data may be accessed by matching the token stored with the health data with the token associated with the patient identity, and then associating the health data with the patient identity.

In an embodiment, the destination system may enable secure access by health operative systems (e.g. pathology, hospital, GP, insurance, etc.). Access may be had securely to the patient data which has been transported and stored in the destination system.

In accordance with a second aspect, the present invention provides an apparatus for securing health data, comprising a processor, memory an operating system supporting computer processes and a network interface for communicating over a communication network, a token preparation process arranged to associate a token with a patient identifier, the network interface being arranged to receive health data having an associated token, a comparison process arranged to compare the token associated with the patient identifier with the token associated with the health data, and, if they match, to associate the patient identifier with the health data.

In accordance with a third aspect, the present invention provides an apparatus for securing data, comprising a processor, memory, a network interface for communicating over a communications network and an operating system supporting computer processes, a health data preparation process arranged to prepare health data for transmission over the communications network via the network interface, the health data preparation process being arranged to associate a token with the health data for transmission.

In accordance with a fourth aspect, the present invention provides a system, comprising an apparatus in accordance with the second aspect and an apparatus in accordance with the third aspect of the invention.

In accordance with a fifth aspect, the present invention comprises a computer program, comprising instructions for controlling a computer to implement an apparatus in accordance with the second aspect or the third aspect of the invention.

In accordance with a sixth aspect, the present invention provides a non-volatile computer-readable medium, providing a computer program in accordance with the fifth aspect of the invention.

In accordance with a seventh aspect, the present invention provides a data signal, comprising a computer program in accordance with the fifth aspect of the invention.

### BRIEF DESCRIPTION OF THE FIGURES

Features and advantages of the present invention will become apparent from the following description of embodiments thereof, by way of example, only, with reference to the accompanying drawings, in which:

FIG. 1 is a schematic diagram of a system in accordance with an embodiment of the present invention;

FIG. 2 is a schematic block diagram of a computer system which may be used to implement an apparatus in the system in accordance with the present invention; and

FIG. 3 is a flow diagram, illustrating operation of a method in accordance with the present invention.

### DETAILED DESCRIPTION OF EMBODIMENTS

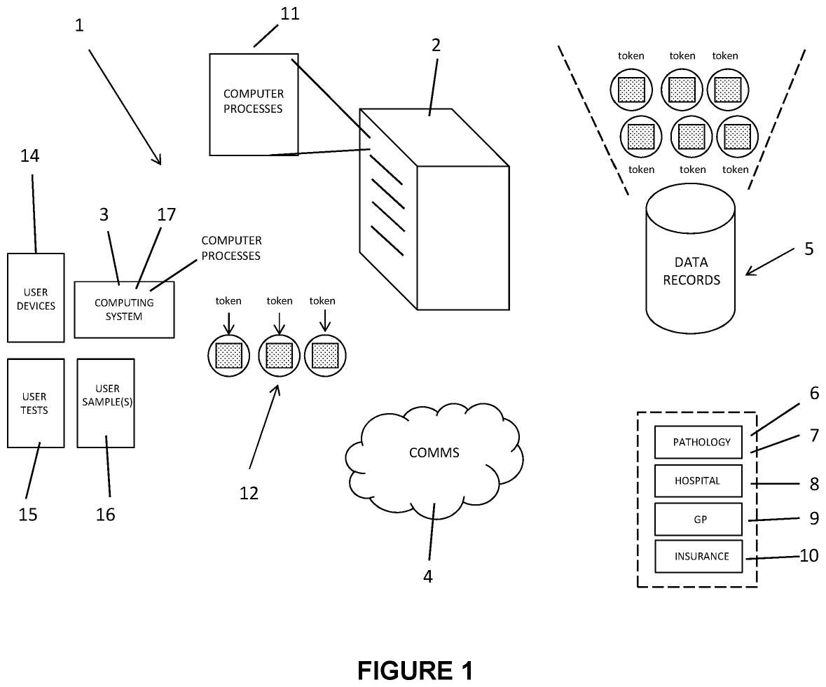

Referring to FIG. 1, a system 1 in accordance with an embodiment of the present invention is illustrated. In this example, the system 1 comprises a destination apparatus 2 and an originating apparatus 3. In this example embodiment, the originating apparatus 3 is a computing system associated with either a patient or a health professional, the computing system 3 is arranged to obtain health data 12 relating to a patient and transmit the health data 12, via a communications network 4, to the destination apparatus 2. In this example, the destination apparatus 2 is a server computing system acting as a central conduit and storage facility for the health data 12.

In this embodiment, the system 1 is arranged to secure the health data by preparing it in an anonymous form. The system is also arranged to associate a token with the health data for transmission with the health data. The system is also arranged to associate a token with a patient identity at the destination apparatus 2. The destination apparatus 2 receives the health data and its associated token. A comparison process arranged to compare the token associated with the health data with the token associated with the patient identity and, if they match, to associate the patient identity with the health data. The health data is therefore transmitted in an “anonymised” fashion across communications network 4. This makes the health data difficult, if not impossible, to hack to obtain relevant information (e.g. health data associated with a patient identity).

The destination system 2 also comprises a database 5 which is able to store health data in data records in the database. In this embodiment, the health data is stored in an anonymous, tokenised form so that if the data is hacked it is difficult for the hacker to obtain any relevant information.

The destination apparatus 2 acts as a secure storage system and conduit and allows secure access to the health data by health operative systems 6. These systems 6 may comprise any authorised system which needs access to patient health data. They may include systems associated with pathology 7, hospitals 8, general practitioners 9, insurance systems 10 (may require health data to provide insurance) or other systems which may require health data.

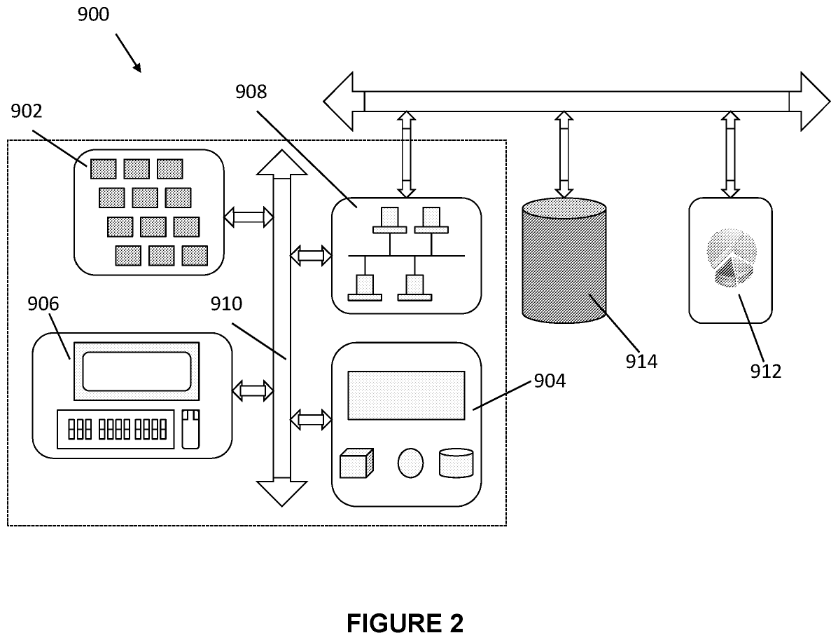

In more detail, referring to FIG. 2, this shows a schematic diagram of a computing system which may be utilised to implement the apparatus and systems illustrated in FIG. 1. The computing system 900 of FIG. 2, for example, may implement the server apparatus 2, or the computing system 3 or computer systems 6. Components of the computing system 900 may be varied, depending upon application in the system 1 and computing systems 3, 2 and 6.

The computer system 900 may be a high performance machine, such as a supercomputer, a desktop workstation or a personal computer, or may be a portable computer such as a laptop or a notebook or may be a distributed computing array or a computer cluster or a networked cluster of computers.

The computer system 900 also comprises a suitable operating system and appropriate software for implementation of embodiments of the present invention.

The computer system 900 comprises one or more data processing units (CPUs) 902; memory 904, which may include volatile or non-volatile memory, such as various types of RAM memories, magnetic discs, optical disks and solid state memories; a user interface 906, which may comprise a monitor, keyboard, mouse and/or touch-screen display; a network or other communication interface 908 for communicating with other computers as well as other devices; and one or more communication busses 910 for interconnecting the different parts of the system 900.

The computer system 900 may also access data stored in a remote database 914 via network interface 908. Remote database 914 may be a distributed database. The database 914 may implement a database 5 shown in FIG. 1.

A computer system for implementing embodiments of the invention is not limited to the computer system described in the preceding paragraphs. Any computer system architecture may be utilised, such as standalone computers, networked computers, dedicated computing devices, handheld devices or any device capable of receiving processing information in accordance with embodiments of the present invention. The architecture may comprise client/server architecture, or any other architecture. The software for implementing embodiments of the invention may be processed by “cloud” computing architecture.

The apparatus 2, the apparatus 3 and the apparatus 6 implement a number of computer processes, supported by the operating systems, which will be described in more detail later on. The computer processes may be implemented as separate modules, which may share common foundations such as routines and sub-routines. The computer processes may be implemented in any suitable way and implementation is not limited to separate modules. Any software/hardware architecture that implements the functionality of the processes as described, may be utilised.

Programmable software may be used to programme processes to implement embodiments of the invention. Programmable hardware may be used to implement embodiments, such as field programmable gate arrays programmable gate arrays and the like. Where software is used to implement embodiments of the invention, the software can be provided on computer readable media, such as discs, or as data signals over networks, such as the internet, or in any other way.

Referring to FIG. 1 again, the server apparatus 2 comprises a processor, memory and operating system which supports computer processes 11. In this embodiment, the computer processes comprise a token preparation process, arranged to generate a token. The token may be a random number, a picture, a code, combination of number, code, picture, alpha numeric, or any other token.

The token preparation process is arranged to associate a token with a patient identifier already stored in system 2. If the patient identifier is not already stored, it may be received by the system 2 over communication network 4. The token is associated with the patient identity, in any convenient way. It may be stored in a database table, for example, together with the patient identity.

The computer processes 11 also comprise a comparison process which is arranged to compare a token received over the communications network together with health data (reference numeral 12), with the token associated with the patient identifier. If the tokens match, the health data 12 is associated in the apparatus 2 with the patient identifier.

A copy of the token is transmitted to the computing system 3. The computing system 3 then associates the token with the health data 12, and transmits the health data with associated token to the apparatus 2, for comparison.

The health data 12 that is transported over the communications network 4 is anonymised. The associated token does not provide any identification of the patient. Identification of the patient is separately carried out the apparatus 2. Any hacker accessing the health data 12 will be unable to find any relevant information relating to the patient.

Apparatus 2 provides a secure system which may provide health data on request to authorised operatives. For example, the apparatus 2 may allow a secure login for health operative systems 6, 7, 8, 9, 10, in order to access health data for particular requested patients that the operatives are authorised for. For example, the pathology system 6 associated with a pathology unit may require health data for assessment and can access the health data securely via the system 2. Any authorised operative may be able to access e.g. operatives associated with hospitals, GP's, insurance or other bodies requiring access to health information.

The health data may be generated in any usual way and then tokenised by the computing system 3. For example, the data may include health tests carried out by a GP or a pathology unit, and the results formed into transportable health data by the computing system 3. The health data may be generated by user devices 14, such as Fitbits™, or any other wearable device. The wearable device may include devices arranged to actively monitor health information, such as blood pressure, heart rate, etc. and associated with a computing system 3 (may be part of a portable device) to prepare the health data for transport and tokenise it. The health data may comprise user sample data 16 (e.g. blood samples) that are prepared by pathology, analysed and then computing system 3 packages and tokenises the health data for a transport.

The health data may be generated in any other way.

In this embodiment, the health data is “packetized”. It is sent in XML form, encrypted, then wrapped in a Network Packet with the token. Packetizing the health data for transport further increases security. In this embodiment, a separate token is generated for each packet, yet further increasing security.

The health data may be formed into separate packets in any desired way. In one embodiment, it may be packetized based on type of health data e.g. heart rate may be packetized separately from blood pressure. In other embodiments it may be packetized merely based on the amount of data e.g. time it was generated. It may be packetized in any other way.

The computing system 3 also supports computer processes 17. The computer processes 17 comprise a health data preparation process which is arranged to prepare the health data 12 for transmission over the communications network 4. The health data preparation process is arranged to associate a token with the health data for transmission.

The apparatus 2 also supports a storage process, which is arranged to securely store health data as data records in database 5. Before storage, each health data packet is associated with a token. The health data is stored in an anonymous form. When the apparatus 2 recovers the health data, it compares the token with a token associated with patient identity (stored elsewhere or securely stored in the same system away from the health data). If the tokens match, then the health data is associated with the patient identity.

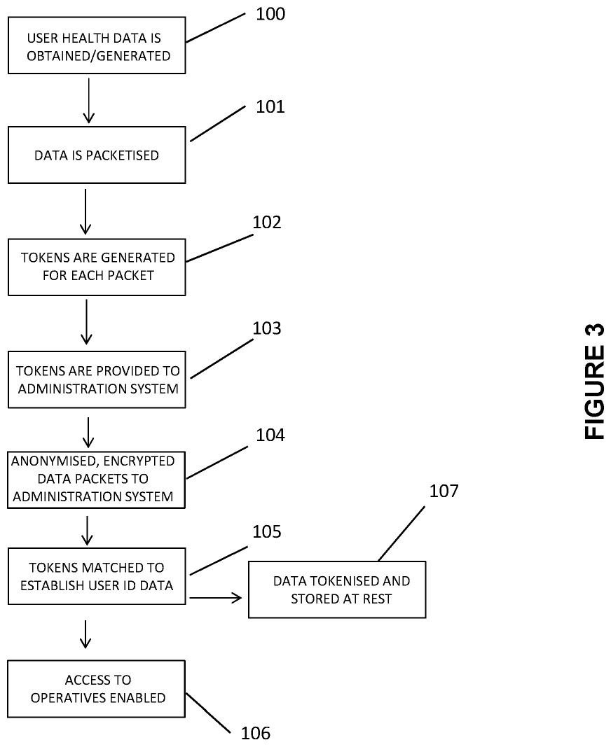

The operation of this embodiment of the system 1 is illustrated in more detail in the flow diagram of FIG. 3.

User health data is obtained or generated (items 14, 15 and 16 of FIG. 1). Data is then packetized by the computer process of computing system 3 (step 101).

Tokens are subsequently generated for each packet (102). In this embodiment, the tokens are generated by the apparatus 2 and transmitted to the computing system 3 to subsequently be associated with the health data packets.

In an alternative embodiment, token generation may occur at the remote computing system 3. Tokens are associated with each health packet generated and, separately, copies of the tokens are transmitted to the apparatus 2 for association with the patient identity. When the health data is received at 2, the tokens are matched and the health data is associated with the patient identity.

Tokens are provided at the apparatus 2 (the administration system) in either of the ways discussed above (either being generated at the apparatus 2 or being provided to the apparatus 2 over the communications network 4 from the computing system 3). Step 103.

The anonymised, encrypted health data packets 12 are transmitted to the administration system 2 (step 104).

At step 105, the tokens are matched to associate the patient identity with the health data.

Access to health operatives systems is enabled (step 106). Access may be any form of secure access to the apparatus 2. For example, the operatives system 6, 7, 8, 9, 10 may have a secure “login” directly to system 2. Other ways of securing access may be enabled.

For storage, data is re-tokenised and stored (step 107). Data is therefore tokenised “in flight” (during transport) and also when it is stored at rest in the database 5. The stored data may later be accessed (step 106) by operative systems.

The patient identifier may be any identifier that could be associated with the patient. It may be name, address, birth, sex, identification of a device worn by the user (e.g. Fitbit number) or any other identifier.

In the above embodiment, the health data is packetized. The invention is not limited to this. In other embodiments, health data may not be packetized, or packetized in a different manner.

In the above embodiment, tokens are generated and then copies of the tokens are generated for transmission to be associated with the patient identity or the health data. The invention is not limited to exact copies of tokens. It may be that mirror images of tokens may be transmitted for matching. Any other means of providing tokens that may be compared in some way, may be utilised.

In the above embodiments, A token is actively disabled once it has been received and matched. This means that the token is “once only” and cannot be used for a subsequent matching. If a hacker does obtain a token, therefore, it will be useless if the token has already been used in a matching process, as the token will be disabled. In an embodiment, a token may also be given a limited lifetime, after which duration they expire. The life time may be in the order of minutes e.g. four to five minutes. It may be any other time period.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A method of securing health data for transport from an originating system to a destination system over a communication network, comprising the steps of preparing the health data in an anonymous form, associating a token with the health data for transmission with the health data, associating a token with a patient identity at the destination system, receiving the health data and its associated token at the destination system, comparing the token associated with the health data with the token associated with the patient identity and, if they match, associating the patient identity with the health data, and wherein the token is a one-use only token.

2. A method in accordance with claim 1, wherein the step of associating a token with the health data comprises the step of generating a token and associating it with the health data.

3. A method in accordance with claim 2, wherein the step of generating the token comprises the step of generating the token when the health data is to be transported.

4. A method in accordance with claim 1, wherein the step of preparing the health data comprises the step of preparing the health data as a plurality of data packets for separate transmission.

5. A method in accordance with claim 4, wherein the step of associating a token with the health data comprises the step of associating a different token with each data packet.

6. A method in accordance with claim 5, wherein the step of associating a token with a patient identity at the destination system, comprises the step of associating a corresponding plurality of tokens with the patient identity as associated with the data packets.

7. A method in accordance with claim 1, comprising the further step of storing the health data, wherein the step of storing comprises anonymising the health data and associating a token with the health data, wherein the health data is stored in tokenised and anonymised form.

8. A method in accordance with claim 7, comprising the further step of accessing the stored data, the step of accessing comprising a step of matching the token(s) associated with the stored health data with token(s) associated with patient identities and stored away from the health data, and if a match occurs then associating the health data with the patient identity.

9. A method in accordance with claim 1, comprising the further step of enabling secure access to the health data by remote operative systems.

10. A method in accordance with claim 1, wherein the step of associating a token with the health data takes place at the originating system, and a copy of the token is transmitted to the destination system for association with the patient identity.

11. A method in accordance with claim 1, wherein the step of associating the token with the patient identity takes place at the destination system, and a copy of the token is transmitted to the originating system for association with the health data.

12. An apparatus for securing health data, comprising a processor, memory an operating system supporting computer processes and a network interface for communicating over a communication network, a token preparation process arranged to associate a token with a patient identifier, the network interface being arranged to receive health data having an associated token, a comparison process arranged to compare the token associated with the patient identifier with the token associated with the health data, and, if they match, to associate the patient identifier with the health data, and wherein the token is a one-use only token.

13. An apparatus in accordance with claim 12, wherein the token preparation process is arranged to generate a token for association with the patient identifier.

14. An apparatus in accordance with claim 13, wherein the token preparation processes is arranged to generate the token when the health data is to be transported.

15. An apparatus in accordance with claim 12, wherein the token preparation process is arranged to transmit a copy of the token via the network interface to an originating system for association with the health data.

16. An apparatus for securing data, comprising a processor, memory, a network interface for communicating over a communications network and an operating system supporting computer processes, a health data preparation process arranged to prepare health data for transmission over the communications network via the network interface, the health data preparation process being arranged to associate a token with the health data for transmission and wherein the token is a one-use only token.

17. An apparatus in accordance with claim 16, the health data preparation process being arranged to prepare the health data as a plurality of data packets for separate transmission.

18. An apparatus in accordance with claim 17, wherein the health data preparation process is arranged to associate a plurality of tokens with the health data, one token for each data packet.

19-25. (canceled)
